SEQUENCE LISTING (3 sequences)

SEQ ID NO: 1 - DNA, 26 bp, Artificial Sequence
  PCR Primer for Exon 5 of the PCMT gene
gttggatgtactggaaaagtcatagg

SEQ ID NO: 2 - DNA, 25 bp, Artificial Sequence
  PCR Primer for Exon 5 of the PCMT gene
caagctgtactctccctgaagacag

SEQ ID NO: 3 - protein, 16 aa, Mus musculus
KERADLIAYLKKATNE

(19) United States
(12) Patent Application Publication
(10) Pub. No.: US 2005/0053984 A1
(43) Pub. Date: Mar. 10, 2005

(54) Isoaspartyl repair

(76) Inventor: Mark J. Mamula, Madison, CT (US)

Correspondence Address: MORGAN, LEWIS & BOCKIUS LLP, 1701 MARKET STREET, PHILADELPHIA, PA, 19103-2921, US

(21) Appl. No.: 10/884,604

(22) Filed: Jul. 2, 2004

Related U.S. Application Data
(63) Continuation of application No. PCT/US03/00186, filed on Nov. 15, 2002.
(60) Provisional application No. 60/426,753, filed on Nov. 15, 2002.
(60) Provisional application No. 60/346,168, filed on Jan. 4, 2002.

(51) Int. Cl. C12Q 1/68

(52) U.S. Cl. 435/6

(57) ABSTRACT

Methods for identifying markers for autoimmune disease which can include comparing the expression of the enzyme “protein carboxyl methyltransferase” (PCMT) in normal subjects to the expression of PCMT in persons diagnosed with an autoimmune disease and methods of diagnosing a subject for an autoimmune disease or a predisposition to an autoimmune disease.

### FIELD OF THE INVENTION

The present invention is in the field of autoimmune disease and immune tolerance.

### BACKGROUND OF THE INVENTION

Many factors can perturb T cell homeostasis. Further, T cell homeostasis is critical in the maintenance of immune tolerance. Defects in the molecules that regulate homeostasis can lead to autoimmune pathology. However, this simple immunologic concept can be complicated by the fact that many self-proteins undergo spontaneous posttranslational modifications that affect their biological functions. This is the case in the spontaneous conversion of aspartyl residues to isoaspartyl residues, a modification occurring at physiological pH and under conditions of cell stress and aging.

Additionally, antigen recognition by the T cell receptor (TCR) in both the thymus and periphery is highly dependent on the conformational structure of the particular antigen based primarily on the amino acid sequence and the stereochemistry of the peptide. In turn, the conformational changes imparted by the TCR determine its fate of thymic selection and for subsequent differentiation and effector functions. However, it is not yet clearly understood what properties of self-peptides or of responding immune cells may allow mechanisms of central or peripheral tolerance to be bypassed in the generation of autoimmunity.

Several recent studies have shown that the posttranslational modification of a single amino acid in a protein/peptide renders an otherwise inert molecule immunogenic.

### SUMMARY OF THE INVENTION

The present invention is directed to a method for identifying markers for autoimmune disease which can include comparing the expression of the enzyme “protein carboxyl methyltransferase” (PCMT) in normal subjects to the expression of PCMT in persons diagnosed with an autoimmune disease and based on the comparative expression, identifying markers that are significantly associated with disease.

In another embodiment, the present invention is directed to a method of diagnosing a subject for an autoimmune disease or a predisposition to an autoimmune disease that comprises comparing the expression of PCMT in the patient with the expression of PCMT in normal subjects, whereby abnormal expression of PCMT in the test subject indicates autoimmune disease or a predisposition to an autoimmune disease.

### BRIEF DESCRIPTION OF THE DRAWING

FIG. 1 shows the structure and formation of isoaspartyl and aspartyl residues.

FIG. 2 shows that isoaspartyl content is elevated in proteins from wild type, PCMT−/−, and PCMT+/− mice.

FIG. 3 shows B cell responses to mitogen and receptor-mediated stimulation.

FIG. 4 shows cell responses to stimulation.

FIG. 5 shows cell responses to stimulation.

FIG. 6 shows that PCMT−/− T cells hyperproliferate in response to ovalbumin restimulation.

FIG. 7 shows that PCMT−/− T cells hyperproliferate in response to ovalbumin restimulation.

FIG. 8 shows Jnk activity in cells from PCMT−/−, PCMT+/− and WT mice.

FIG. 9 shows an increase of phosphorylation of signaling molecules in anti-CD3/CD28 mAb stimulated lymph node cells.

FIG. 10 shows an analysis of Jnk activity in PCMT−/− CD4⁺ T cells.

FIG. 11 shows that the mature lymphoid compartment is most likely selected on a unique collection of isoaspartyl modified self-peptides in PCMT−/− mice.

FIG. 12 shows indirect immunofluorescence using sera from congenic mice reconstituted with either wild-type littermate or PCMT−/− bone marrow.

FIG. 13 shows the histopathology of kidney sections from bone marrow recipient mice.

### DETAILED DESCRIPTION OF THE INVENTION

The immune system has developed a number of mechanisms to avoid autoreactivity, including pathways of central and peripheral tolerance. However, the presence of autoimmune diseases suggests that these pathways are not perfect in the regulation of immune responses. Certainly the introduction of several posttranslational modifications into self-proteins can affect the immunogenicity of an otherwise ignored protein. In this regard, it has been demonstrated that the posttranslational modification of an aspartyl residue to an isoaspartyl residue in self-peptides breaks tolerance. Further, isoaspartyl accumulation induces aberrant T cell signaling with hyperproliferation and the initiation of autoreactive antibody production.

The formation of isoaspartyl modifications is one of the single major mechanisms for the degradation of cellular proteins under physiologic conditions of temperature and pH. Indeed, eukaryotes have evolved the enzyme, protein carboxyl methyltransferase (PCMT), to selectively methylate the α-carboxyl residue of isoaspartyls in attempts to repair deleterious modifications. PCMT enzyme functions have also been identified in plant cells, fungi, insects and bacteria, with the latter observation supporting the presence of isoaspartyl residues in recombinant proteins expressed in prokaryotes. S-adenosyl methionine serves as the methyl donor to this enzymatic repair reaction. The endoplasmic retention signal found on PCMT suggests that isoaspartyl repair functions can be initiated as newly synthesized proteins exit the endoplasmic reticulum. In spite of these efforts for cellular repair of isoaspartyl modifications, the less-than-perfect efficiency of PCMT allows modified proteins to escape into the cellular milieu particularly under conditions of aging and cellular stresses such as heat shock.

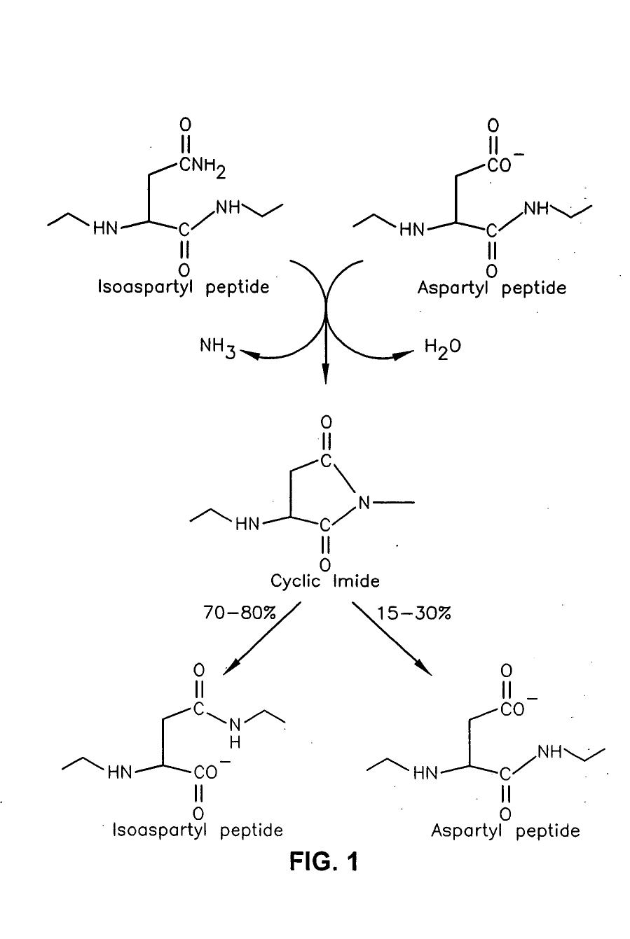

As shown in FIG. 1, isoaspartyl residues are generated by the spontaneous deamidation of asparagine or isomerization of aspartic acid. The resulting succinimide intermediate is relatively unstable and is spontaneously hydrolyzed under physiological conditions at either carbonyl group, giving a mixture of normal aspartyl protein (30% product) or isoaspartyl residues (70% product). The later modification is the result of the peptide bond through the side chain beta carboxyl, leaving the alpha carboxyl as the free carbon.

In other areas, isoaspartyl modifications sometimes affect the biological functions of proteins in which they exist. For example, isoaspartyl modifications have been observed in age-damaged calmodulin at Asp/Asn-Gly sequences in the Ca⁺⁺ binding domain. Nearly 80% of its biological activity is destroyed by this modification which can then be restored by repair with PCMT. Additionally, as set forth herein, there is no evidence indicating that cellular death pathways in lymphocytes are affected by the accumulation of these modifications. Isoaspartyl and/or deamidated residues have also been found to reduce some properties of CD4 in binding to gp120 of HIV and of the biological activity of murine IL-1.

The consequences of isoaspartyl accumulation in cellular proteins, and thus cell function, is best demonstrated in the PCMT−/− mouse. Although there is an increase in isoaspartyl residues in cell lysates from all the lymphoid organs in the PCMT−/− mouse, the most dramatic effect of these modifications appears to be in the T cell compartment. The accumulation of isoaspartyl residues has no apparent effect on lymphoid development, as there were normal proportions of cells within the spleen, lymph nodes, and thymus. However, T cell stimulation by either mitogen or TCR was clearly increased in PCMT−/− mice as compared to wild-type mice, with PCMT+/− T cell proliferation falling somewhere between these two groups. The fact that PCMT+/− mice have half the intracellular concentrations of PCMT provides an explanation for the intermediate proliferate phenotype of heterozygous mice that exists.

The enhanced T cell proliferation in response to antigen was not accompanied by increased activation of lymphocytes in vivo or by decreased apoptosis, suggesting a defect in T cell signaling. The pattern of hyperproliferation in PCMT−/− T cells was similar to that reported in Jnk1−/− mice, also with no apparent effect on lymphocyte development. An analysis of amino acid sequences indicates that Jnk1 does contain three susceptible sites with Asp/Asn-Gly or Asp/Asn-Ser in which isoaspartyl formation can spontaneously arise. Even though a decrease in Jnk activity in PCMT−/− T cells as compared to wild-type T cells was not observed, the pattern of anti-CD28 mAb stimulation and the decrease in Th2 polarization did not correspond exactly with that seen in Jnk1−/− T cells.

In an effort to understand the effect of these hyperproliferating T cells on in vivo biological immune responses, wild-type congenic mice with PCMT−/− bone marrow were reconstituted. The rationale for these studies lies in the observation that cells with uncontrolled or hyperproliferative phenotypes may elicit autoimmune responses. For example, the MRL lpr/lpr mouse, a murine model of human systemic lupus erythematosus (SLE), exhibits spontaneous T cell activation coincident with the expression of autoantibodies and autoimmune pathology.

In an analogous manner, mice reconstituted with PCMT−/− bone marrow exhibiting hyperproliferative T cells demonstrated signs of autoantibody production over time. Although PCMT−/− bone marrow was selected on a “normal” unmodified repertoire of self-peptides, there is a strong likelihood that altered thresholds of T cell activation in these mice can affect thymic selection. While the peripheral cell subsets appear normal in number, the appearance of spontaneous autoimmunity suggests that either the specificity or ability to be activated by self-antigens can be severely affected in mice bearing lymphoid cells with isoaspartyl modifications. Once in the periphery, T cells originating from PCMT−/− bone marrow have a lowered threshold for activation such that self-peptides normally maintaining peripheral tolerance are now able to induce T cell activation. It is clear from our studies that much lower amounts of a nominal foreign antigen are needed to induce proliferation in PCMT−/− mice as compared to wild-type T cells.

It is becoming increasingly clear that posttranslational modifications can break tolerance to self-antigens. In several models of autoimmune diseases, the presence of posttranslationally modified self-antigens induces an immune response. Amino terminal acetylation of myelin basic peptide 1-11, or the phosphorylation of α-B-crystallin have both been shown to induce disease or T cell responses, respectively, in murine models of experimental autoimmune encephalomyelitis (EAE). In addition, autoantibodies from patients with SLE have been shown to immunoprecipitate phosphorylated proteins from Jurkat cells undergoing apoptosis. Finally, T cell hybridomas derived from mice immunized with type II collagen react to glycosylated peptides of collagen, while sera from rheumatoid arthritis patients positive for antifilaggrin Ab specifically recognize deaminated human filaggrin.

As described in more detail below, an accumulation of isoaspartyl residues in cells alters the effector function of lymphocytes leading to autoimmunity. It is important to note that the bone marrow reconstitution studies were designed to identify the in vivo biologic function of these modified cells in the context of an otherwise normal peptide environment in the thymus and periphery. The use of PCMT−/− cells allowed for the artificially provocation of intracellular modifications that would otherwise accumulate spontaneously in aged and stressed cells. Accordingly, isoaspartyl formation, and its deleterious effect on lymphocyte function, may be one triggering event by which spontaneous autoimmunity arises.

Isolation and Assay of PCMT

The isospartyl repair enzyme protein carboxylmethyltransferase (PCMT) activity can be assayed directly out of peripheral blood red blood cells (RBCs). In brief, heparinized blood is withdrawn from normal individuals and test subjects. RBCs are isolated, washed in PBS and lysed by 5 cycles of freeze thawing. PCMT can then be purified by precipitation in 55% ammonium sulfate and by affinity chromatography on columns composed of rabbit anti-human PCMT. For comparative purposes, a positive control PCMT can include recombinant human PCMT.

PCMT from 10 normal controls, patient specimens, positive control PCMT, and blank negative controls can be assayed by a vapor diffusion assay. This assay measures the methyl transferase activity of PCMT by the uptake of methyl groups from S-adenosyl [3H-methyl]-L-methionine into the methyl accepting substrate isoaspartyl modified cytochrome c (hereinafter “cyt c”). Normal PCMT, patient PCMT samples, and positive control PCMT can be incubated with cyt c modified at one site with isoaspartyl as the methyl acceptor substrate or the aspartyl form of cyt c, a non-methyl accepting substrate. Accordingly, release of 3H-methanol, as a measure of PCMT activity, can then be measured by liquid scintillation chromatography.

Levels/Amounts of RBC PCMT Measured by ELISA.

PCMT protein levels can be measured from RBC lysates by ELISA. Specifically, RBC lysates can be prepared as above and adsorbed to the surface of microtiter plates. Plates can be blocked with 1% bovine serum albumin and incubated with rabbit anti-human PCMT or mouse monoclonal antibodies specific for human PCMT. Plates can be washed and bound antibody can be detected by secondary anti-rabbit Ig or anti-mouse Ig linked to alkaline phosphatase followed by incubation with the AP substrate, paranitrophenyl phosphate (pNPP). Levels of PCMT can be compared to a standard curve generated by the titration of purified recombinant human PCMT at concentrations from 0.01 μg/ml to 100 μg/ml. Abnormal values from patient specimens can be determined as having PCMT protein levels at least two standard deviations (2SD) below or above the mean of normal controls. However, it should be noted that certain tissues are known to contain different baseline levels of PCMT, for example brain, testes, and heart have higher levels. It should be further noted that PCMT is primarily in the cytosolic fractions of cells although some membrane PCMT has been reported in brain cells. RBCs are used as the typical source of PCMT since these cells do not exhibit de novo protein synthesis.

Based on a comparative expression, one can identify markers that are significantly associated with disease and specific activity of PCMT of all samples can be assessed by the above-described procedure. Aberrant PCMT can be defined as levels that are greater than 2SD below or above the mean of about 10 normal controls. A mean of 556+/−43 units and 558+/−42 units of PCMT activity/ml erythrocytes has been established for normal males and females, respectively.

Indicia and Markers of Autoimmunity

Accordingly, a predisposition for autoimmunity in this assay can be based on levels of PCMT specific activity falling >2SD below the mean of normal controls. Further, PCMT deficiency in murine models has been associated with autoimmunity and in early fatality related to brain seizures of unknown etiology.

The method can also be based on a comparison of genotypic markers (i.e., one or more polymorphisms in the PCMT gene or in a different genetic loci.), and/or phenotypic markers (i.e., differences in the protein sequence (including, e.g., positional isomers), and/or the enzymatic activity of the PCMT.

The PCMT gene is located to human chromosome bands 6q24-6q25. Known PCMT polymorphisms in introns are found at residues 22, 119, and 205. While thermal stability of PCMT varies according to these described polymorphisms, there is no apparent affect on PCMT specific activity, nor have polymorphisms been unambiguously linked with any clinical disease entity.

The link between PCMT polymorphisms and disease has been discussed in various references. Additionally, a 50% decrease in PCMT activity has been found in the tubulin of the brains of patients with mesial temporal lobe epilepsy and it is hypothesized to be linked to that clinical syndrome. In support of such indicia, and as described above, mice made genetically deficient in PCMT typically die within 6 weeks of age due to epileptic-like seizures.

Although there are no known biological syndromes related to the overexpression or over activity of PCMT, a PCMT transgenic mouse crossed with a PCMT deficient mouse can reverse the early death and seizures found in PCMT−/− animals. These studies demonstrate the efficacy of restoring defective PCMT enzyme functions by gene therapy approaches to reverse clinical disease. Finally, the over expression of PCMT has been shown to increase the lifespan of drosophila by nearly 40%.

Characterization of Polymorphisms

Single stranded conformational polymorphisms (SSCP) of PCMT in humans can be characterized by PCR. The procedure can requires 200-300 μl of whole blood that can be easily acquired by finger stick blood drawing from patients and controls. RBCs are lysed in buffer and genomic DNA can be purified by conventional approaches using chloroform:isoamyl alcohol (24:1). Genomic DNA can then be amplified with primers specific for exon 5 of the PCMT gene known to encode the major polymorphism at residue 119 and yielding a 118 bp product after amplification

| | |
|---|---|
| (5′GTTGGATGTACTGGAAAAGTCATAGG-3′ | (SEQ ID NO:1) |
| and | |
| 5′-CAAGCTGTACTCTCCCTGAAGACAG-3′). | (SEQ ID NO:2) |

Exon 5 contains the major polymorphism found in human PCMT. In a similar manner, exons 2 and 7 can be PCR amplified with specific primers. Amplified DNA fragments can be nucleotide sequenced with commercially available sequencing kits.

Diagnosis of Autoimmune Disease

The present invention also includes a method of diagnosing a subject for an autoimmune disease or a predisposition to an autoimmune disease that involves comparing the expression of PCMT in the patient with the expression of PCMT in normal subjects, whereby abnormal expression of PCMT in the test subject indicates autoimmune disease or a predisposition to an autoimmune disease. Such a comparison can be based on genotypic markers (i.e., one or more polymorphisms in the PCMT gene or in a different genetic loci.), and/or phenotypic markers (i.e., differences in the protein sequence (including, e.g., positional isomers), and/or the enzymatic activity of the PCMT.

As described above, PCMT function can be assayed by the vapor diffusion method. “Normal” values have been reported previously as described above and we would define “abnormal” as +/−2SD away from “normals.”

The present invention can also include a marker for autoimmune disease identified by the process set forth above.

The present invention also includes a diagnostic assay method which comprises screening subjects for the marker associated with disease, as described above.

Functional Assays

The diagnostic assay for the function of PCMT can include commercially available 3H—S adenosyl methionine and peptides consisting of isoaspartyl and aspartyl residues. Additionally, in one embodiment, the present invention employs a scintillation counter to quantify the release of 3H methanol, as described above. In some embodiments, peptides of mouse cyt c, p88-104 (KERADLIAYLKKATNE (SEQ ID NO:3)) with and without isoaspartyl at position 5 as substrates for methylation by PCMT, can be used. Additionally, as a positive control, recombinant PCMT, either human or rat, can be used.

It should be noted that a commercial kit is available from Promega (Isoquant) that tests for the presence of isoaspartyl in candidate proteins. The Promega kit uses control monomer isoaspartyl peptide of DSIP (Delta Sleep Inducing Protein). For the analysis of PCMT function, the test can be reported as units/mg protein or units/ml blood, as described above. Similar to above, abnormals would be described as +/−2SD away from the mean of “normals.”

Similar to above, patient RBCs can be used as the source of PCMT.

Methods of Treatment

The present invention also includes a method of treating an autoimmune disease in a patient which comprises restoring PCMT activity to normal levels.

As cited above, animal models demonstrate that restoring PCMT functions can resolve seizures and prolong life in PCMT deficient animals. This provides the basis and/or rationale for reconstituting PCMT in enzyme deficient patients with autoimmunity (or seizures). The basis for restoring PCMT in autoimmunity is that PCMT deficiency causes aberrant, hyperproliferative T cell responses. Furthermore, it is known that gene defects that cause T cell hyperproliferation also lead to lupus like autoimmunity. Additionally, overexpression or underexpression of PCMT can also be a factor in erythematosus, multiple sclerosis, rheumatoid arthritis and type II diabetes because these diseases are fundamentally similar in action to each other and to lupus in that each disease involves aberrant T cell function. It is because PCMT deficiencies can lead to aberrant T cell responses and aberrant T cell responses can lead to diseases including, but not limited to, erythematosus, multiple sclerosis, rheumatoid arthritis, type II diabetes, and lupus, that these and other diseases which result from aberrant T cell responses can be grouped together for purposes of the present invention.

Accordingly, PCMT reconstitution can utilize conventional gene therapy approaches in adenoviral vectors. Specifically, an adenovirus vector can be employed to restore PCMT in cultured neurons and to the brains of fetal PCMT−/− mice. More specifically, the present invention can clone the human PCMT gene into adenovirus for administration to humans. Treatment can also include the administration of wild-type PCMT to a patient.

In addition, the present invention provides for modulation of PCMT activity. Specifically, incubating living cells in AdOX (adenosine dialdehyde) commercially available from Sigma Corp, interferes with methyl transferase functions in general, and can block PCMT function in particular. This can lead to intracellular increases in isoaspartyl content.

As shown in FIG. 1, isoaspartyl residue formation occurs at physiologic temperature and pH via the spontaneous intramolecular deamidation at Asn-X linkages or by the isomerization of Asp-X linkages. Hydrolysis of the cyclic imide intermediate results in the formation of the isoaspartyl form of protein (approximately 70-80% of the product) and of the normal aspartyl form (approximately 20-30% of the end product). The isoaspartyl form results in the peptide bond occurring through the β-carbonyl rather than the normal α-carbonyl group.

FIG. 7 shows that PCMT−/− T cells hyperproliferate in response to ovalbumin restimulation. PCMT−/−, PCMT+/−, and WT mice were immunized with 50 μg OVA emulsified in complete Freund's adjuvant (CFA). Ten days later, purified T cells were isolated from draining lymph nodes and co-cultured with irradiated syngenic splenocytes plus titrating amounts of OVA. PPD controls ranged from 3310-8261 cpm. Error bars represent SD, representative of three experiments.

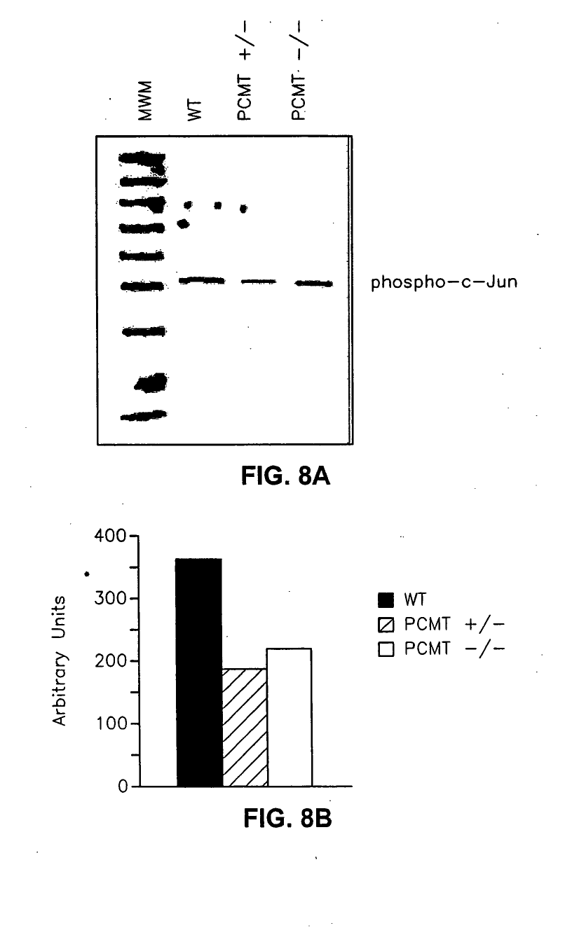

FIG. 8 shows aberrant JNK kinase activity in PCMT−/− CD4⁺ T cells. CD4⁺ T cells (1×10⁶) from PCMT−/−, PCMT+/−, and WT mice were stimulated with anti-CD3 and anti-CD28 mAb for 44 h. Cell lysates were prepared, normalized for protein concentration and immunoprecipitated with c-Jun Sepharose. The kinase assay was performed and the presence of phosphorylated c-Jun was determined by Western Blotting. FIG. 8A) represents a Western Blot of phosphorylated c-Jun. FIG. 8B) represents a densitometric analysis of the Western Blot. Results are representative of three experiments.

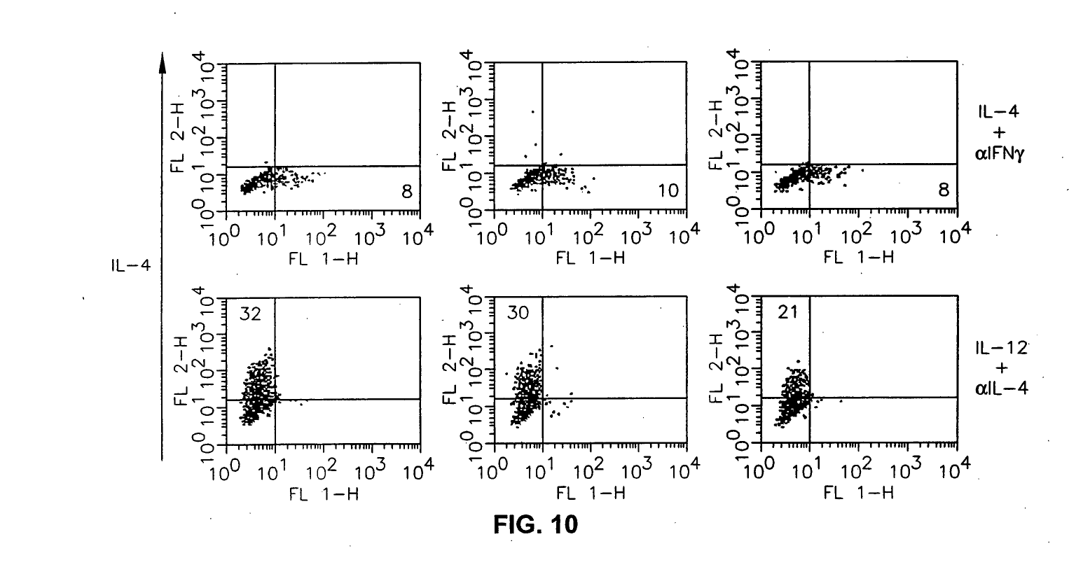

As shown in FIG. 10, there can be Th1/Th2 differentiation in PCMT−/− CD4⁺ T cells. Purified naïve CD4⁺ T cells from PCMT−/−, PCMT+/−, and WT mice were stimulated with anti-CD3 mAb plus anti-CD28 mAb in the presence of either IL-4 or IL-12. Four days after culture, the cells were restimulated with anti-CD3 mAb and cells were analyzed by flow cytometry for intracellular cytokines IL-4 or IFNγ.

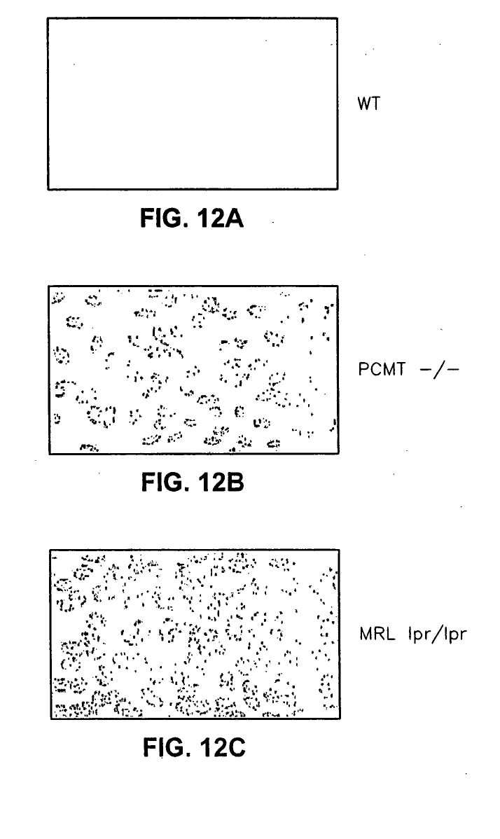

FIG. 12 shows indirect immunofluorescence using sera from congenic mice reconstituted with either wild-type littermate or PCMT−/− bone marrow. Sera were collected from congenic mice reconstituted with (A) wild-type or (B) PCMT−/− bone marrow and examined for the presence of antinuclear antibodies (ANA) on Hep-2 cell substrates. (C) Positive control serum from MRL lpr/lpr mouse.

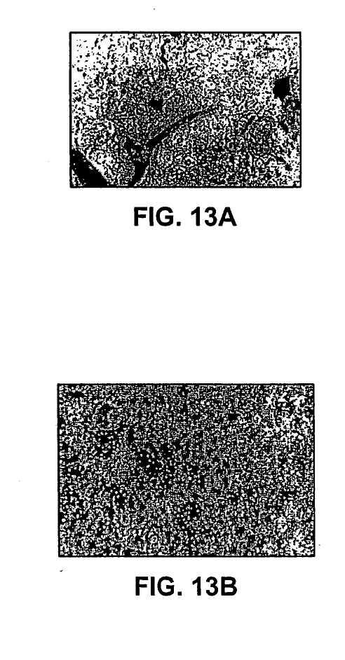

As described in the examples below, FIG. 13 shows the histopathology of kidney sections from bone marrow recipient mice. Kidney sections were obtained from congenic C57Bl/6 mice 7 to 9 months after receiving PCMT−/− syngenic bone marrow (panel A) or wild-type bone marrow (panel B).

The following examples are given to illustrate the present invention. It should be understood, however, that the invention is not to be limited to the specific conditions or details described in these examples. Throughout the specification, any and all references to a publicly available document, including but not limited to a U.S. patent, are specifically incorporated by reference.

### Example 1

### PCMT Deficient Mice

PCMT+/− mice were obtained from the University of California, Los Angeles, and were generated by inserting a neo cassette into exon one of the PCMT gene. PCMT−/− mice were obtained by intercrossing PCMT+/− C57BL/6 mice. Mice were screened for the presence of the neo cassette and the absence of the PCMT gene by PCR analysis of tail DNA. Primer sequences were: PCMT forward 5′-gcagcgacggcagtaacagc-3′, PCMT reverse 5′cgcatcgagcgagcacgtactcgg-3′, neo forward 5′-gcacgaggaagcggtcagcccattc-3′ and neo reverse 5′-cgcatcgagcgagcacgtactcgg-3′. The PCMT−/− mice were used at 4-6 weeks of age. C57BL/6 mice (National Cancer Institute, Frederick, Md.) and B6.SJL-ptprcaPep3B/Boy/J mice (Jackson Laboratory, Bar Harbor, Me.) were used at 4-6 weeks of age. Unless otherwise stated, wild-type mice are age-matched littermates.

### Example 2

### Measurement of Isoaspartyl Protein in Cells

Spleen, lymph node, and thymus cells were resuspended in immunoprecipitation buffer (10 mM Tris pH 8.0, 500 mM NaCl, and 0.1% NP-40) and sonicated. Cell lysates were centrifuged 15,000×g for 10 min and the total protein concentration of the supernatants determined using the DC Protein Assay per manufacturer's instructions (BioRad, Hercules, Calif.). Isoaspartyl content of spleen, lymph node and thymus cell lysates was measured using a PCMT vapor diffusion assay (ISOQUANT Protein Deamidation Detection Kit, Promega, Madison, Wis.). Briefly, 2 μg of cell lysate was incubated with 1 μCi S-adenosyl-L-[methyl-³H] methionine (Amersham Life Science, Piscataway, N.J.) and PCMT in sodium phosphate buffer at 30° C. for 30 min. The methyl-transfer reaction was terminated with a basic pH stop solution. Fifty microliters of the reaction cocktail was spotted onto a sponge insert attached to a scintillation vial cap and placed on a scintillant-filled vial for 1 hr. at 37° C. The sponge-containing caps were removed and replaced with new caps, and radioactivity measured with a scintillation counter (Beckman, Fullerton, Calif.). PBS served as the negative control and the isoaspartyl Delta Sleep Inducing Peptide (IsoAsp-DSIP) served as the positive control.

### Example 3

### B Cell Proliferation Assays

B cells were isolated from spleen by negative selection using the MACS system (Miltenyi Biotec, Auburn, Calif.). The resulting cell population was 80-85% pure as determined by flow cytometry. Cells were resuspended in Click's medium+5% FCS supplemented with 2 mM L-glutamine, 0.1 mM β-mercaptoethanol and antibiotics (100 U/ml penicillin/streptomycin, 50 μg/ml gentamicin) then plated at 1×10⁵ cells/well in 0.2 ml volume in 96-well flat bottom microtiter plates with either 1 μg/ml LPS (Sigma), 40 μg/ml anti-IgM Ab (Jackson ImmunoResearch Laboratories, Inc., West Grove, Pa.), 2.5 μg/ml anti-CD40 Ab (clone HM 40-3, PharMingen, San Diego, Calif.) plus 20 ng/ml IL-4 (R& D Systems, Minneapolis, Minn.) or media alone. Cells were incubated for 48 hours at 37° C., 5% CO₂, after which cells were pulsed with 1 μCi ³H-thymidine (ICN Chemicals, Irvine, Calif.) for 18 hours prior to being harvested onto filters with a semiautomatic cell harvester. Radioactivity was counted with a Betaplate liquid scintillation counter (LKB/Wallac Inc., Gaithersburg, Md.).

### Example 4

### Mitogen and anti-CD3 mAb Stimulation

Spleen cells were suspended in Click's medium+5% FCS supplemented with 2 mM L-glutamine, 0.1 mM β-mercaptoethanol and antibiotics (about 100 U/ml penicillin/streptomycin, 50 μg/ml gentamicin). Cells were plated at 5×10⁵ cells/well in 0.2 ml volume in 96-well flat bottom microtiter plates with or without 2 μg/ml Con A (Sigma, St. Louis, Mo.). For anti-CD3 mAb stimulation of splenocytes, wells of a 96-well U bottom microtiter plate were coated with or without 30 μl of a 2.5 μg/ml concentration anti-CD3 mAb (clone 145-2C11, hamster IgG) (PharMingen, San Diego, Calif.) in PBS and incubated at 37° C. for 1 h. Wells were washed 3× with PBS prior to the addition of 2.5×10⁵ cells/well in 0.2 ml volume. After incubating 2-3 days at 37° C., 5% CO₂, cells were pulsed with 1 μCi ³H-thymidine/well (ICN Chemicals, Irvine, Calif.) for 18 hr. and harvested onto filters with a semiautomatic cell harvester. Radioactivity was counted with a Betaplate liquid scintillation counter (LKB/Wallac Inc., Gaithersburg, Md.). CD3/CD28 stimulation was done by coating 96-well flat bottom plates with 0.5 □g anti-CD3 mAb overnight at 4° C. Wells were washed, and 0.1 μg of anti-CD28 mAb (clone 37.51, PharMingen) was added to the appropriate wells prior to the addition of 1×10⁵ purified CD4⁺ T cells. CD4⁺ T cells were obtained by negative selection of spleen and lymph node cells using the MACS system (Miltenyi Biotec, Auburn, Calif.). T cell purity was 80% as determined by FACS analysis.

### Example 5

### T Lymphocyte Proliferation Assays

Wild-type C57BL/6, and PCMT−/− C57BL/6, and PCMT+/− C5BL/6 mice were immunized with 50 μg OVA (Sigma, St. Louis, Mo.) emulsified 1:1 in CFA (Sigma, St. Louis, Mo.) at the base of the tail and hind footpad. Ten days later, draining lymph node T lymphocytes were isolated by negative selection using a cocktail of antibodies against B220 (TIB 146), Mac-1 (TIB 128), and anti-I-A^{b} (Y3-JP) for 1 hr. on ice. Cells were washed and incubated with sheep anti-mouse/rat Ig coated magnetic beads (PerSeptive Biosystems, Framingham, Mass.) at a ratio of 5:1 (beads: cells) for 1 hr. at 4° C. Enriched T cells (82-88% pure) were separated from non-T cells with a magnet. T lymphocytes (1×10⁵) were resuspended in Clicks medium+5% FCS supplemented with L-glutamine, β-mercaptoethanol, antibiotics and cultured with irradiated (2500 rads) C57BL/6 spleen cells (5×10⁵) with or without antigen. PPD (M. tuberculosis H37 RA, Difco Laboratories, Detroit, Mich.) served as a positive control. After 3 days, cells were pulsed and harvested as described above.

### Example 6

### Antibodies and Flow Cytometry

All antibodies were purchased from PharMingen (San Diego, Calif.) and included: FITC anti-CD4 (RM4-5, rat IgG2a), PE anti-CD62L (MEL-14, rat IgG2a), Cy-Chome CD44 (IM-7, rat IgG2b), FITC anti-CD8 (53-6.7, rat IgG2a), PE anti-CD4 (H129.19, rat IgG2b), Cy-Chome B220 (RA3-6B2, rat IgG2a), FITC anti-1-Ab (AF6-120.1, mouse IgG2a), PE anti-CD11b (M1/70, IgG2b), FITC anti-CD45.1 (A20, mouse IgG2a), and FITC anti-CD45.2 (104, mouse IgG2a). Cell surface staining with optimal concentrations of fluorochome-conjugated mAb was performed on 5-10×10⁵ cells in 0.2 ml PBS+1% BSA+0.1% NaN₃ for 30 min at 4° C. Cells were washed 3× and fixed in PBS+1% paraformaldehyde. Samples were analyzed on a FACSCalibur (BDIS, Mountain View, Calif.).

### Example 7

### Determination of Activation-Induced Cell Death

CD4⁺ T cells from PCMT−/− or wild-type mice were purified by negative selection using the MACS system (Miltenyi Biotec, Auburn, Calif.), plated at 4×10⁶ cells/well of a 6 well plate in the presence of 2.5 μg/ml Con A and incubated 2 days at 37° C., 5% CO₂. Cells were washed extensively and dead cells removed by Ficoll gradient separation (Amersham Pharmacia, Piscataway, N.J.). Cells were plated in wells coated with anti-CD3 mAb plus 50 U/ml IL-2 and incubated for 48 hrs. before staining for apoptosis. Cells were washed 2× with binding buffer (10 mM HEPES, pH 7.4, 140 mM NaCl, 2.5 mM CaCl₂), and 1×10⁵ cells incubated with 5 μl each AnnexinV-PE and 7-amino-actinomycin D (7-AAD) for 15 min at room temperature in the dark. Four hundred microliters of binding buffer was added to each sample and analyzed by FACS within 1 h. Cells undergoing apoptosis stain Annexin V⁺ 7-AAD⁻. Non-stimulated cells served as negative controls for apoptosis.

### Example 8

### Phosphorylation Analysis of TCR Stimulated PCMT−/−Lymph Node Cells

Lymph node cells were stimulated with plate bound anti-CD3 mAb (10 μg/ml)+anti-CD28 mAb (5 μg/ml) for 2 min in Click's medium without fetal calf serum. The reaction was stopped by putting the cells on ice, the cells spun down and the pellet homogenized in ice-cold lysis buffer [20 mM MOPS (pH 7.0), 2 mM EGTA, 5 mM EDTA, 30 mM NaF, 40 mM β-glycerophosphate (pH 7.4), 10 mM sodium pyrophosphate, 2 mM sodium orthovanadate, 1 mM PMSF, 3 mM benzamidine, 5 μM pepstatin A, 10 μM leupeptin, 0.5% Nonidet P-40]. The homogenate was centrifuged at 14,000 rpm for 15 min and the resulting supernatant removed and immediately assayed for protein concentration by the Bradford assay (Bio-Rad Laboratories, Inc, Hercules, Calif.). The final concentration of protein was adjusted to 0.5 mg/ml in SDS-PAGE sample buffer. Three hundred fifty micrograms of protein was subjected to the Kinetworks™ Phopsho-site Screen (KPSS-1.1) and analysis of phosphoproteins done per the manufacturer's specifications (Kinexus Bioinformatics Corporation, Vancouver, BC, Canada). Results were expressed in both a gel format and as densitometry readings. According to the manufacturer's protocol, changes greater than 25% are considered significant. Percentage change in band intensity was calculated as [(PCMT−/− trace quantity-wild-type trace quantity)/PCMT−/− trace quantity]×100.

### Example 9

### Generation of PCMT−/− Bone Barrow Chimeras

Bone marrow was isolated aseptically from PCMT−/− or wild-type littermates, washed in PBS, and resuspended in PBS+1 mM HEPES at a concentration of 1×10⁸ cells/ml. B6.SJL-ptprcaPep3B/BoyJ/J (CD45.1) congenic mice were sublethally irradiated (750 rads) using a ¹³⁷Cs irradiator. Mice were rested several hours prior to i.v. injection of 20×10⁶ cells. Four to six weeks after bone marrow transfer, bone marrow reconstitution was confirmed by FACS analysis of peripheral blood cells for the CD45.2 marker as described above.

### Example 10

### Indirect Immunofluorescence (ANA)

Indirect immunofluorescence assays for antinuclear antibodies (ANA) were performed using commercially available substrates (Quidel, San Diego, Calif.). Briefly, 30 μl of a 1:100 dilution of mouse sera were placed on slides coated with human epithelial cells (HEp-2) and incubated for 2 hrs. at room temperature. Slides were washed 10 min in PBS, and then individual wells incubated with 30 μl of a 1:50 dilution FITC anti-mouse IgG Ab (Sigma, St. Louis, Mo.) and incubated in the dark for 2 h. Slides were washed 10 min in PBS, and examined by fluorescence microscopy. Serum from a single MRL lpr/lpr mouse positive for ANA served as the positive control.

### Example 11

### Anti-dsDNA Antibody ELISA

Anti-dsDNA autoantibody was examined using a commercially available ELISA (Sanofi-Pasteur Diagnostics, Chaska, Minn.). Briefly, DNA coated wells were blocked with PBS+5% BSA for 1 hr. at room temperature, then washed 3× with PBS. Fifty microliters of 1:100 dilutions of mouse sera were added to each well and incubated 2 hrs. at room temperature. Wells were washed 3× with PBS, followed by the addition of 50 μl of a 1:1000 dilution goat anti-mouse IgG alkaline phosphatase (Southern Biotechnology Associates, Birmingham, Ala.) for 2 hrs. at room temperature. Wells were washed 5× with PBS and 50 μl of p-nitrophenylphosphate substrate (Sigma, St. Louis, Mo.) was added to each well. The plates were read at 405 nm on a spectrophotometric ELISA reader at various timepoints. Experimental sera were normalized to a single MRL lpr/lpr positive control serum used in every assay.

### Example 12

### Kidney Pathology

Kidneys from PCMT−/− and wild-type bone marrow reconstituted mice were collected at 7-9 months post bone marrow transfer and immediately immersed in 10% formalin (Fisher, Pittsburgh, Pa.). Thin sections and hematoxylin and eosin staining were performed by the Yale Dermatopathology Laboratory. Blinded samples were examined for pathology at 20× magnification.

### Example 13

### Isoaspartyl Cell Content in Lymphoid Organs

Mice lacking PCMT were used to define the effect of isoaspartyl modifications on self-proteins in cellular immune functions. PCMT−/− mice are unable to repair posttranslational modifications resulting in enhanced isoaspartyl modifications in tissues. All groups of mice examined had virtually identical distributions of cell types (B and T cell subsets) in the thymus, lymph node, and spleen (data not shown). An analysis of B and T cell activation states by flow cytometry also revealed no distinct differences among any of the mouse groups. Taken together, these observations indicate that no significant defects or differences exist in central T cell selection or in the development of T cell subsets in the periphery of PCMT−/− mice as compared to wild-type mice.

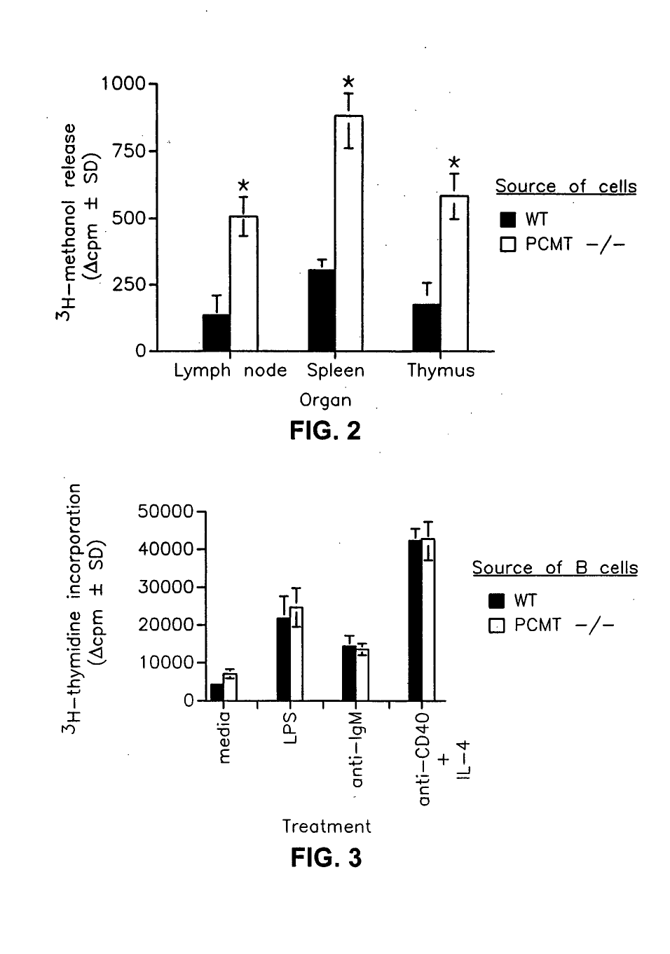

The isoaspartyl content of cell lysates from spleen, lymph node and thymus was quantitated from wild-type and PCMT−/− mice using a vapor diffusion assay that specifically identifies isoaspartyl posttranslational modifications, as described above. As illustrated in FIG. 2, PCMT−/− cell lysates had significantly increased levels of intracellular isoaspartyl modifications in the spleen (p<0.004), lymph node (p<0.001), and thymus (p<0.020) as compared to wild-type mice. More specifically, FIG. 2 shows that the isoaspartyl content is elevated in proteins from PCMT−/− mice. The results represent the difference between the experimental counts and background cpm. Background was 864 cpm and the DSIP positive control was 11,638 cpm.

### Example 14

### PCMT−/− Mice Can Have Increased T cell Proliferative Responses to Mitogen or Receptor-Mediated Stimulation

Initial analyses of immune function were performed in PCMT−/− mice to determine the effect of isoaspartyl accumulation on B and T lymphocyte functions. An analysis of B lymphocyte functions revealed no apparent differences between PCMT−/− mice and wild-type mice with respect to peripheral B cell numbers, activation phenotypes, or distribution (data not shown). As indicated in FIG. 3, B cell responses to mitogen and receptor-mediated stimulation were nearly identical between PCMT−/− and wild-type mice, where LPS, anti-IgM and anti-CD40+IL-4 were used.

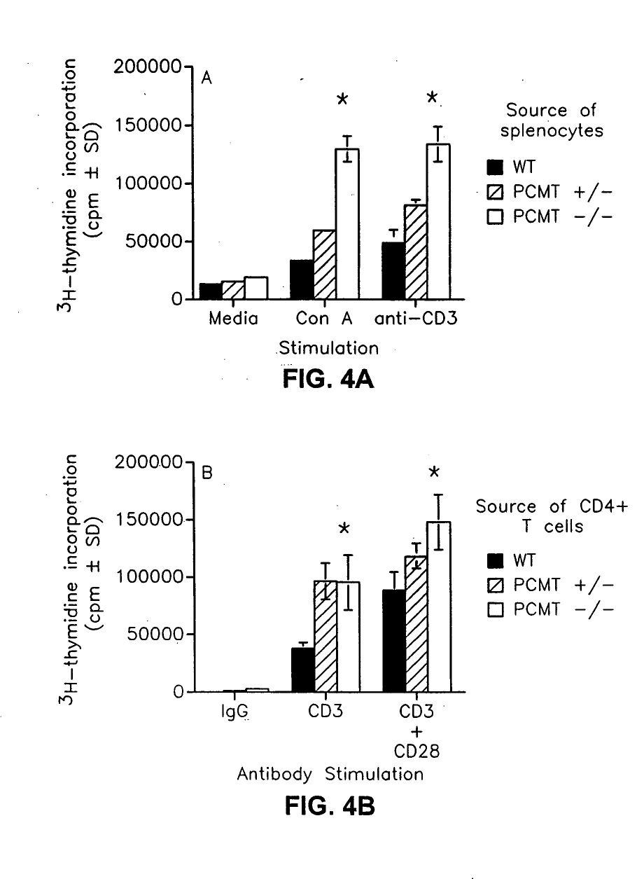

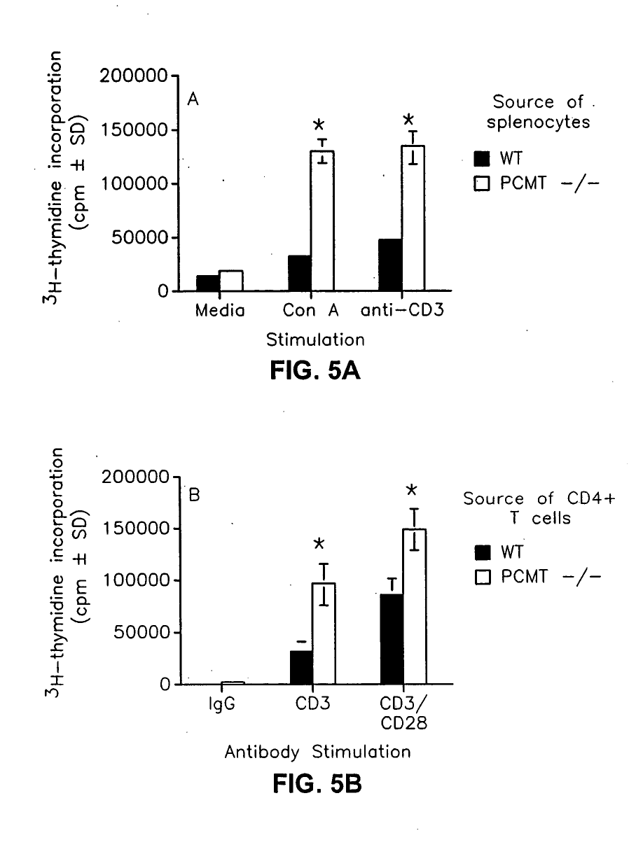

For the generation of the data in FIGS. 4A and 5A splenocytes from PCMT−/−, PCMT+/−, and WT mice were incubated with or without Con A or plate-bound anti-CD3 mAb 2-3 days before harvesting. For the generation of data in FIGS. 4B and 5B, purified CD4⁺ T cells from PCMT−/−, PCMT+/−, and WT mice were incubated with plate-bound anti-CD3 mAb with or without anti-CD28 mAb for 72 h before harvesting. Error bars represent the SD (absence of bars indicate error too small to be observed in this figure). A “*” represents a significant difference as compared to WT cells. Results are representative of three experiments.

Differences between PCMT−/− and wild-type mice were observed in the T cell compartment. There was a significant increase in PCMT−/− splenocyte proliferation in response to Con A as compared to cells from wild-type animals (p<0.002; FIGS. 4A 5A). Consistent with the above observation, anti-CD3 mAb receptor-mediated stimulation of T cells also resulted in a significant increase in proliferative signaling in PCMT−/− splenocytes as compared to wild-type splenocytes (p<0.001; FIGS. 4A and 5A). Anti-CD3 mAb stimulation of purified PCMT−/− CD4⁺ T cells in the presence of anti-CD28 mAb further confirmed that the hyperproliferation was indeed in the T cell compartment (FIGS. 4B and 5B). The addition of anti-CD28 mAb to the cultures enhanced proliferation in all groups, including in PCMT−/− lymphocytes (FIGS. 4A and 5B). FIGS. 5A and 5B also show that the PCMT+/−cells exhibit responses that typically fall between the PCMT−/− and wild-type cellular responses.

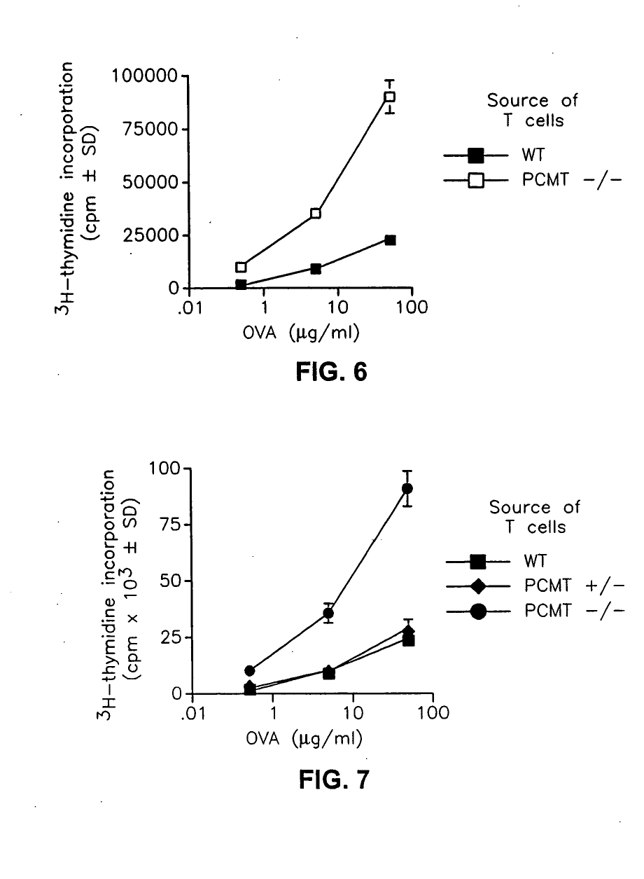

Having demonstrated that PCMT−/− splenocytes and CD4⁺ T cells were hyperresponsive to mitogen and receptor-mediated stimulation in vitro, it was next determined whether T cells in lymph nodes from PCMT−/− mice would respond in a similar manner in vivo to a nominal foreign antigen. For these studies, PCMT−/− and control littermate mice were immunized with OVA-CFA for 10 days followed by an analysis of recall responses of purified T cells stimulated with OVA antigen. As seen in FIG. 6, T cells from PCMT−/− mice responded with up to four-fold greater proliferation as compared to T cells from wild-type mice. The enhanced T cell response of PCMT−/− mice was not due to differences in APC function, since no differences were found in the ability of PCMT−/− splenocytes to present OVA as compared to wild-type splenocytes (data not shown).

For comparative purposes, as shown in FIG. 7, PCMT−/−, PCMT+/−, and WT mice were immunized with 50 μg OVA emulsified in CFA. Ten days later, purified T cells were isolated from draining lymph nodes and co-cultured with irradiated syngeneic splenocytes plus titrating amounts of OVA. PPD controls ranged from 3310-8261 cpm. Error bars represent SD, representative of three experiments. Similar to above, PCMT−/− mice responded with up to four-fold greater proliferation as compared to T cells from wild-type mice and PCMT+/−mice.

A hyperproliferative phenotype of T cells may cause abnormal thymic selection to occur or allow abnormally activated or memory T cell populations to arise over time in vivo. Flow cytometry analysis revealed that there were no significant differences in CD4 or CD8 populations in the spleen or lymph node of PCMT−/− mice as compared to wild-type littermates (data not shown). Furthermore, the majority of CD4⁺ and CD8⁺ cells in both organs was of a naïve phenotype (CD62L^{hi}CD44^{lo}) and resembled those populations found in age matched wild-type mice (78% vs 78%). Thymic selection appeared fundamentally normal by the similarity of double and single positive cells in the thymus of PCMT−/− mice compared to those seen in wild-type littermates (CD4⁺ CD8⁺; 87% in both PCMT−/− and wild-type mice). Finally, antigen presenting cells (CD11b⁺) and resident B cell populations (B220⁺) in the spleen were equivalent between all mouse groups (data not shown). Additionally, there were no detectable differences in the expression of MHC class II on either the B220⁺ or CD11b⁺ positive populations.

We next examined the possibility that the increased hyperproliferation in PCMT−/− T cells reflected a decrease in apoptosis of these cells after activation-induced cell death (AICD). To address this possibility, CD4⁺ T cells from PCMT−/− and wild-type mice were induced to undergo AICD followed by staining with Annexin V and 7-amino-actinomycin D (7-AAD). Cells undergoing apoptosis were found to be Annexin V⁺ 7-AAD⁻. CD4⁺ T cells from both PCMT−/− mice and wild-type mice exhibited similar apoptotic populations upon stimulation with anti-CD3 mAb (45% PCMT−/− vs. 46% wild-type Annexin V⁺ 7-AAD⁻; data not shown). Therefore, abnormal proliferative functions of T cells bearing increased posttranslational modifications are not due to any defects in the death pathway of cells.

### Example 15

### Aberrant JNK Kinase Activity in PCMT−/− CD4⁺ T Cells

As shown in FIG. 8, CD4⁺ T cells (1×10⁶) from PCMT−/−, PCMT+/−, and WT mice were stimulated with anti-CD3 and anti-CD28 mAb for 44 hours. Jnk activity was assayed using a nonradioactive (chemiluminescent) SAPK/Jnk assay kit (New England Biolabs, Inc, Beverly, Mass.) per manufacturer's instructions. Briefly, cultured cells were washed 2× with ice-cold PBS, then resuspended in 1× Cell Lysis Buffer (20 mM Tris, pH 7.4, 150 mM NaCl, imM EDTA, 1 mM EGTA, 1% Triton, 2.5 mM NA pyrophosphate, 1 mM β-glycerolphosphate, 1 mM Na₃VO₄, 1 μg/ml leupeptin, 1 mM PMSF) for 5 min prior to sonication. The resulting lysate was centrifuged, and the supernatant used in the assay. A c-Jun fusion protein bound to glutathione sepharose beads was used to pull down Jnk from cell lysates. The kinase reaction was carried out using ATP, and the phosphorylated c-Jun detected by western blotting using an antibody specific for phosphorylated c-Jun. The membrane was developed using the Phototope-HRP Western Detection Kit (New England Biolab, Inc., Beverly, Mass.) and exposed to x-ray film. Densitometric readings were performed using the Gel Doc system (BioRad, Hercules, Calif.). The results are shown in FIG. 8A, a western blot of phosphorylated c-Jun and FIG. 8B, a densitometric analysis of Western Blot. Results are representative of three experiments.

### Example 16

### Increased Phosphorylation of Signaling Molecules in Anti-CD3/CD28 mAb Stimulated Lymph Node Cells

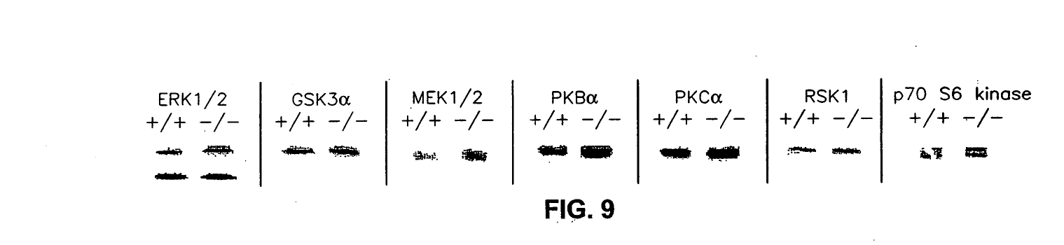

Lymph node cells from either PCMT−/− mice or wild-type littermates were stimulated with anti-CD3/CD28 mAb for 2 min at 37° C. Cell lysates were then prepared for analysis of phosphoproteins by the Kinetworks™ Phospho-site Screen. As shown in FIG. 9, there was an increased phosphorylation of signaling molecules in anti-CD3/CD28 mAb stimulated lymph node cells.

The hyperproliferative phenotype of PCMT−/− T cells upon TCR stimulation suggests certain signaling molecules are affected by the accumulation of isoaspartic acid residues within the cell. Cell lysates prepared from anti-CD3/CD28 mAb stimulated lymph node cells from PCMT−/− and wild-type mice were analyzed by the Kinetworks™ Phospho-site Screen to detect differences in the phosphorylation of proteins involved in TCR signaling. As listed in Table 1, a number of signaling proteins were hyperphosphorylated in PCMT−/− lymph nodes as compared to wild-type lymph nodes. CD3 ligation resulted in the hyperphosphorylation of MAPK pathway members mitogen-activated protein kinase/extracellular-regulated kinase 1/2 (MEK1/2), extracelluar signal-regulated kinase (ERK) 1/2, and 90 kDa ribosomal S6 kinase (RSK) 1. There was no difference in the phosphorylation status of two other members of the MAPK pathway, c-Jun N-terminal kinase (JNK) and p38 (data not shown). Protein kinase C (PKC)α, also activated upon TCR/CD28 stimulation, was hyperphosphorylated in PCMT−/− lymph node cells. CD28 stimulation has been shown to induce protein kinase Bα (PKBα) phosphorylation and indeed PKBα, as well as one of its substrates, glycogen synthase kinase 3α (GSK3α), was hyperphosphorylated in anti-CD3/CD28 mAb stimulated PCMT−/− cells. This is in addition to another CD28 regulated molecule, p70 S6 kinase, which is also hyperphosphorylated in PCMT−/− lymph node cells. Together, these data suggest that numerous signaling molecules within the TCR/CD28 signaling cascade are affected by isoaspartyl accumulation.

Table 1. Differential phosphorylation of proteins in PCMT−/− and wild-type (WT) lymph node cells after anti-CD3 mAb+anti-CD28 mAb stimulation as determined by Kinetworks™ Phospho-site screening.

| | Trace Quantity | | % |
|---|---|---|---|
| | PCMT | | Increase |
| Protein Name (Abbreviation) | −/− | WT | over WT |
| Extracellular regulated kinase 1 (ERK1) | 7174 | 4686 | 35 |
| Extracellular regulated kinase 2 (ERK2) | 9693 | 6535 | 33 |
| Glycogen synthase kinase 3 α (GSK3α) | 8722 | 6029 | 31 |
| MAP kinase kinase ½ (MEK ½) | 1408 | 530 | 62 |
| Protein kinase B α (PKBα) | 16099 | 10043 | 38 |
| Protein kinase C α (PKCα) | 19994 | 12986 | 35 |
| Ribosomal S6 kinase 1 (RSK1) | 4884 | 2613 | 46 |
| S6 kinase p70 (p70 S6K) | 1316 | 556 | 58 |

### EXAMPLE 17

### Aberrant Cell Signaling and Differentiation in PCMT−/− T Cells

PCMT−/− T cell hyperproliferation was not explained by the presence of abnormal lymphocyte populations, abnormal levels of activated lymphocytes, or by aberrant levels of apoptosis, leaving open the possibility that the presence of isoaspartyl residues might affect TCR signaling pathways. The hyperproliferative phenotype in response to anti-CD3/CD28 mAb stimulation indicated a potential defect in Jnk1 signaling. As found in FIG. 10, analysis of Jnk activity in PCMT−/− CD4⁺ T cells showed a two-fold decrease in Jnk activity as compared to wild-type mice. Since Jnk1 has been shown to negatively regulate the production of Th2 cytokines, Th1 and Th2 cytokine expression from CD4⁺ T cells cultured with anti-CD3/CD28 mAb under Th1 conditions (IL-12+anti-IL-4 mAb) or Th2 conditions (IL-4+anti-IFNγ mAb) was analyzed. PCMT−/− CD4⁺ T cells differentiated to Th1 cells to a similar extent as did CD4⁺ T cells from wild-type mice. However, when cultured under Th2 conditions, Th2 polarization was inhibited by 11% in PCMT−/− mice (32% vs. 21%) as compared to wild-type mice, as shown in FIG. 10. Cells from PCMT+/−mice did not differ significantly in the production of IL-4 or IFNγ from wild-type mice under any of the conditions examined, as also shown in FIG. 10.

### Example 18

### Mice Reconstituted with PCMT−/− Bone Marrow Develop Antibodies to Self-Antigens

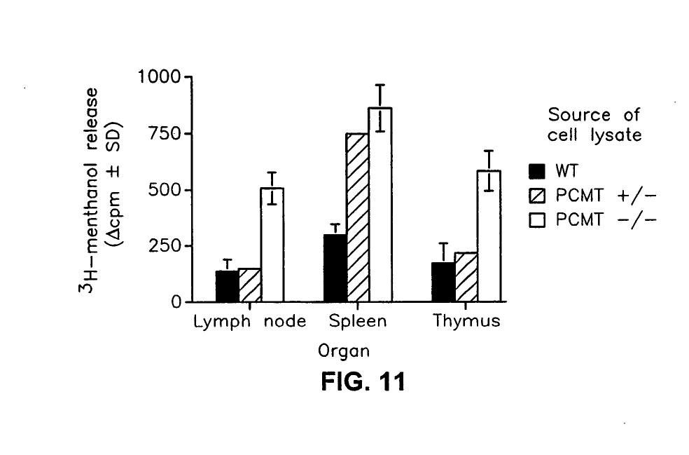

The immune status of PCMT−/− lymphoid cells in the “normal” antigenic environment of a wild-type syngenic mouse was then examined. The rationale for these studies lies in the fact that the mature lymphoid compartment is most likely selected on a unique collection of isoaspartyl modified self-peptides in PCMT−/− mice (shown by FIG. 11). Further, it has been observed in humans with SLE and in murine models of SLE that T cells exhibit hyperresponsiveness to antigenic stimulation in a manner much like that found in PCMT−/− T cells. Human and murine SLE is marked by the appearance of autoantibodies to a variety of intracellular macromolecules including nucleosomes, dsDNA, and ribonucleoproteins. Accordingy, whether this abnormal T cell phenotype could lead to other autoimmune phenomenon in vivo was examined.

Since PCMT−/− mice only survive approximately 6 weeks, long term studies of immune responses in irradiated wild-type mice receiving PCMT−/− bone marrow were studied.

For these studies, CD45.1 bearing congenic mice were reconstituted with CD45.2-marked PCMT−/− bone marrow, allowing the fate of transferred cells to be tracked. In preliminary studies, it was first examined whether sera from bone marrow reconstituted mice would bind intracellular proteins in indirect immunofluorescence assays (IIF). Sera from mice reconstituted with wild-type littermate bone marrow failed to exhibit antinuclear antibodies (ANA) by indirect immunofluorescence similar to what is seen with serum from unmanipulated wild-type mice, shown in FIG. 12A. In contrast, sera from mice reconstituted with PCMT−/− bone marrow showed positive staining of nuclear antigens with some staining of the nucleoli, the representative pattern in FIG. 12B. In attempts to identify specific intranuclear components bound by autoimmune sera in PCMT−/− reconstituted mice, ELISA was performed with dsDNA as the antigenic substrate. FIG. 12C represents a positive control serum from MRL lpr/lpr mouse.

As shown in Table 2, mice reconstituted with PCMT−/− bone marrow generated elevated levels of anti-DNA autoantibodies as compared to mice receiving wild-type bone marrow. There did not appear to be a perfect correlation between a positive ANA and the presence of anti-dsDNA antibodies indicating that other intranuclear proteins are targets of the autoantibody response.

TABLE 2
Autoreactivity in wild-type mice reconstituted with
PCMT−/− bone marrow.

| | Bone Marrow | | dsDNA Ab ELISA |
|---|---|---|---|
| Mouse | Transferred | Positive ANA | Endpoint Titer |
| 1 | PCMT−/− | +3 | 1:3200 |
| 2 | PCMT−/− | +2 | 1:6400 |
| 3 | PCMT−/− | +1 | 1:100 |
| 4 | PCMT−/− | +2 | 1:100 |
| 5 | PCMT−/− | +1 | 1:100 |
| 6 | WT | − | 1:100 |
| 7 | WT | +/− | 1:100 |

### EXAMPLE 19

### Kidney Pathology is Observed in Wild-Type Mice Receiving PCMT−/− Bone Marrow

After antinuclear antibodies were found in recipients of PCMT−/− bone marrow, the pathology in kidney sections of host animals was examined. Some recipients of PCMT−/− bone marrow exhibited severe renal pathology as found in FIG. 13A. The pathology was marked by perivascular cellular infiltration, infiltration of myointimal regions with lymphocytes, focal areas of necrosis, and endovasculitis. In contrast, recipients of wild-type bone marrow were unremarkable and primarily normal in renal architecture (FIG. 13B).

Each reference cited herein is hereby incorporated by reference in its entirety.

1. A method for identifying markers for autoimmune disease which comprises:
(a) comparing the expression of PCMT in normal subjects to the expression of PCMT in persons diagnosed with an autoimmune disease
(b) based on said comparative expression, identifying markers that are significantly associated with disease.

2. The method of claim 1, wherein the comparison is based on genotypic markers.

3. The method of claim 1, wherein the comparison is based on phenotypic markers.

4. The method of claim 1, wherein the autoimmune disease is a disease selected from the group consisting of lupus erythematosus, multiple sclerosis, rheumatoid arthritis, type II diabetes.

5. A method of diagnosing a subject for an autoimmune disease or a predisposition to an autoimmune disease that comprises comparing the expression of PCMT in the patient with the expression of PCMT in normal subjects, whereby abnormal expression of PCMT in the test subject indicates autoimmune disease or a predisposition to an autoimmune disease.

6. The method of claim 5, wherein the comparison is based on genotypic markers.

7. The method of claim 5, wherein the comparison is based on phenotypic markers.

8. The method of claim 5, wherein the autoimmune disease is a disease selected from the group consisting of lupus erythematosus, multiple sclerosis, rheumatoid arthritis, type II diabetes.

9. A marker for autoimmune disease identified by the methods of claim 1.

10. A diagnostic assay method which comprises screening subjects for the marker of claim 1.

11. A diagnostic assay kit comprising reagents for the identification of the marker of claim 1.

12. A method of treating an autoimmune disease in a patient which comprises restoring PCMT activity to normal levels.

13. The method of claim 12 wherein the PCMT activity is restored by: gene therapy or the administration of wild type PCMT.
